(19) United States
(12) Patent Application Publication
(10) Pub. No.: US 2024/0081895 A1
(43) Pub. Date: Mar. 14, 2024

(54) ROTARY SHAVER ARRANGEMENT FOR A SURGICAL INSTRUMENT

(71) Applicant: GYRUS MEDICAL LIMITED, Cardiff (GB)

(72) Inventor: James Alan DICKSON, Cardiff (GB)

(73) Assignee: GYRUS MEDICAL LIMITED, Cardiff (GB)

(21) Appl. No.: 18/367,186

(22) Filed: Sep. 12, 2023

Related U.S. Application Data
(60) Provisional application No. 63/406,530, filed on Sep. 14, 2022.

(51) Int. Cl. A61B 18/14; A61B 17/32; A61L 29/10; A61L 29/14

(52) U.S. Cl. CPC A61B 18/148; A61B 17/320016; A61L 29/106; A61L 29/14; A61B 2018/00101

(57) ABSTRACT

A rotary shaver component for a surgical instrument or an end effector for an electrosurgical instrument which has a thermally insulating component on an inner surface of a tubular member. The thermally insulating component is particularly useful for electrosurgical instruments which combine rotary shaver arrangements and RF electrode arrangements, where suction is used to remove RF heated saline from the surgical site. Without the presence of the thermally insulating component, there is a risk of burning the patient if the RF heated saline becomes too hot as the electrosurgical instrument may not be adequately insulated. The thermally insulating component prevents or reduces the effect of hot saline heating at least a portion of the inner tubular member, thereby preventing or reducing damage to non-target tissue during use of the radio frequency function.

### TECHNICAL FIELD

Embodiments of the present disclosure described herein relate to surgical devices, and in particular to a rotary shaver component and arrangement for a surgical instrument and an end effector for an electrosurgical instrument.

### BACKGROUND TO THE INVENTION AND PRIOR ART

Electrosurgical instruments provide advantages over traditional surgical instruments in that they can be used for coagulation and tissue sealing purposes. Surgical apparatus used to shave, cut, resect, abrade and/or remove tissue, bone and/or other bodily materials are known. Such surgical apparatus can include a cutting surface, such as a rotating blade disposed on an elongated inner tubular member (or shaft) that is rotated within an elongated outer tubular member (or shaft), each tubular member having a cutting window. The inner and outer tubular members together form a surgical cutting instrument or unit. In general, the elongated outer tubular member includes a distal end defining an opening or cutting window disposed at a side of the distal end of the outer tubular member. The cutting window of the outer tubular member exposes the cutting surface of the inner tubular member (located at a side of the distal end of the inner tubular member) to tissue, bone and/or any other bodily materials to be removed. A powered handpiece is used to rotate the inner tubular member with respect to the outer tubular member while an outer tubular member hub (connected to the proximal end of the outer tubular member) is fixed to the handpiece and an inner tubular member hub (connected to the proximal end of the inner tubular member) is loosely held in place by the powered handpiece.

In some instruments the inner tubular member is hollow and has a cutting window on a side surface of its distal end such that tissue, bone, etc. will be cut or shaved as the cutting window of the inner tubular member aligns with and then becomes misaligned with the cutting window of the outer tubular member as the inner tubular member is rotated within the outer tubular member. In this regard, it can be said that the cutting device removes small pieces of the bone, tissue, etc. as the inner tubular member is rotated within the outer tubular member.

In some instruments a vacuum is applied through the inner tubular member such that the bodily material that is to be cut, shaved, etc. is drawn into the windows of the inner and outer tubular members when those windows become aligned, thereby facilitating the cutting, shaving, etc. of the tissue, which then travels through the inner tubular member due to the suction.

Many times during surgery, the surgeon wishes to apply RF energy to either coagulate bleeding vessels, or ablate tissue in the surgical site without performing cutting with a shaver instrument. This usually is done by withdrawing the surgical instrument and inserting a dedicated RF wand device (for example, an RF ablation wand). However, exchanging the surgical tool for the dedicated RF device is time-consuming. Furthermore, insertion and removal of instruments into the patient can cause trauma and irritation to the passage of the patient, and thus it is desirable to minimize the number of times that surgical instruments need to be withdrawn and inserted/reinserted into the patient.

### SUMMARY OF THE DISCLOSURE

Combining a shaver device with an RF device is not straightforward. The RF plasma generated by arthroscopic RF ablation probes causes heating of the surrounding saline. In the case of suction-capable RF probes, this heated saline passes through the shaft and handle of the device, leaving via outflow tubing at the proximal end. The construction of a typical RF suction probe shaft is such that there is significant thermal insulation present between the heated saline and the outermost surface of the shaft—the surface most likely to be in contact with the skin of the patient during an arthroscopic procedure. This thermal insulation allows the suction of heated saline while ensuring the risk of a burn to the patient remains low. FIG. 1 shows this typical shaft construction which has significant thermal insulation present between the tubular member and the outermost surface of the instrument in the form of an air gap and multiple polymer layers. On the other hand, arthroscopic shaver probes do not typically require this thermal insulation, as the saline being drawn into the device is generally no greater than ambient temperature, and there is therefore no risk of a burn due to heated saline.

Embodiments of the present invention aim to combine these two very different devices, a shaver device and an RF suction device. Due to the geometric constraints of such a combination, this effectively results in a typical shaver construction with the additional function and components of an RF probe. The RF shaver must therefore manage the risks of heated saline.

The present disclosure addresses the above problem of managing risks of heated saline by providing a thermal insulation component within the electrosurgical instrument construction. In this manner, the insulation component insulates the inside portion (i.e., the inner surface) of the inner blade which in use may be in direct contact with non-target tissue on its outside face (i.e., its outer surface).

In view of the above, from a first aspect, the present disclosure relates to a rotary shaver component for a surgical instrument, the rotary shaver component comprising: a tubular member providing a central suction lumen, the tubular member comprising a first cutting window at the distal end thereof; and a thermal insulation component provided at the distal end of the tubular member, the thermal insulation component being provided on at least a portion of an inner surface of the tubular member located opposite the first cutting window (opposite refers to the first cutting window being located at a first circumferential location of the tubular member and the thermal insulation component being provided on at least a portion of the inner surface which is at a second circumferential location of the tubular member, wherein the first and second circumferential locations are opposite each other).

Several advantages are obtained from embodiments according to the above-described aspect. For example, such a component could be used in an RF shaver instrument where the component is used as an inner tubular member. Insulating the inner tubular member from hot saline which passes through the central suction lumen of the instrument during usage of the RF functionality prevents the outer surface of the inner tubular member (which may be in contact with non-target tissue) from reaching dangerously high temperatures.

From a second aspect, the present disclosure relates to a rotary shaver arrangement for a surgical instrument, the rotary shaver arrangement comprising: an outer tubular member having a central passageway, the tubular member comprising a second cutting window at the distal end thereof; the rotary shaver component as described immediately above, wherein the tubular member is an inner tubular member rotatably mounted in the central passageway of the outer tubular member and the first and second cutting windows align when the inner tubular member is rotated to a first position.

From a third aspect, the present disclosure relates to an end effector for an electrosurgical instrument, the end effector comprising: a tubular member providing a central suction lumen; a lateral facing radio frequency active electrode mounted at the distal end of the tubular member, the electrode comprising a suction aperture, the suction aperture being in fluid communication with the central suction lumen; a thermal insulation component provided at the distal end of the tubular member, the thermal insulation component being provided on at least a portion of an inner surface of the tubular member, wherein, when the radio frequency active electrode is in use, the portion of the inner surface is located opposite the lateral facing radio frequency active electrode (opposite refers to the electrode being located at a first circumferential location of the tubular member and the thermal insulation component being provided on at least a portion of the inner surface which is at a second circumferential location of the tubular member, wherein the first and second circumferential locations are opposite each other).

As described herein, the heat shield of the present disclosure may be used in any RF suction probe, with or without rotary shaver functionality. Having insulation on the inner surface of the tubular member prevents high temperatures on the outer surface of the tubular member and thus prevents or reduces injury to non-target patient tissue in contact with the outer surface of the tubular member. This may be complimented by insulation external to the tubular member if the structure of the instrument permits it.

In some embodiments, the end effector further comprises an outer tubular member having a central passageway; wherein the tubular member is an inner tubular member rotatably mounted in the central passageway of the outer tubular member, the inner tubular member comprises a first cutting window at the distal end thereof, the outer tubular member comprises a second cutting window at the distal end thereof, such that the first and second cutting windows align when the inner tubular member is rotated to a first position, and wherein the portion of the inner surface is located opposite the first cutting window. In this embodiment, both the first cutting window and the electrode are located at the first circumferential location of the inner tubular member, and the thermal insulation component is provided on at least a portion of the inner surface which is at the second circumferential location of the inner tubular member, wherein the first and second circumferential locations are opposite each other.

This embodiment includes rotary shaver functionality. The thermal insulation component is particularly useful for electrosurgical instruments which combine an RF suction probe and a rotary shaver device because insulation external to the inner tubular member is difficult to implement, as explained herein.

In some embodiments of any of the aspects described above, the arrangement is such that, when in use, rotation of the inner tubular member within the outer tubular member causes a tissue cutting action of the first cutting window interacting with the second cutting window.

In some embodiments of any of the aspects described above, the thermal insulation component is such that the distal end of the thermal insulation component is thicker than the proximal end of the thermal insulation component. This is advantageous as such a configuration provides sufficient thermal insulation where needed while leaving adequate space for cut tissue to be suctioned along the central suction lumen and thus removed from the surgical site without blocking the lumen.

In some embodiments of any of the aspects described above, the thermal insulation component is shaped such that it provides a sloped surface from the distal end of the inner tubular member such that the height of the thermal insulation component is greater at the distal end of the thermal insulation component than at the proximal end of the thermal insulation component. Again, this is advantageous because it leaves adequate space for cut tissue to be suctioned along the central suction lumen.

In some embodiments of any of the aspects described above, the thermal insulation component comprises a thermally insulating coating on at least the portion of the inner surface of the inner tubular member. The thickness of the coating can be chosen based on the thermal properties of the material of the coating. For example, in one advantageous embodiment, the coating may be made from a highly thermally insulating material, and as such, the coating can be a thin coating which does not take up much space in the central suction lumen, thereby leaving more space for cut tissue to be suctioned along the central suction lumen.

In some embodiments of any of the aspects described above, the thermal insulation component comprises a ceramic material. The ceramic material may comprise Alumina and/or Zirconia.

In some embodiments of any of the aspects described above, the thermal insulation component comprises a heat resistant polymer.

In some embodiments of any of the aspects described above, the thermal insulation component is fitted to at least the portion of the inner surface by one or more of the following: interference fit, adhesive and snap fit.

In some embodiments of any of the aspects described above, the thermal insulating component is a solid component.

In some embodiments of any of the aspects described above, the thermal insulating component is a hollow component.

In some embodiments of any of the aspects described above, the thermal insulating component comprises a heat shield surface and an air gap, wherein the air gap is positioned between the heat shield surface and the inner surface of the tubular member located opposite the first cutting window. This is advantageous because air is a better thermal insulator than most solid materials, and air is also lighter than a solid material so would not upset the rotational balance of the blade as much when spinning.

In some embodiments of any of the aspects described above, the thermal insulation component is fitted to at least the portion of the inner surface by welding. For example, the heat shield surface may be fixed to the inner surface by welding the ends or edges of the heat shield surface to the inner surface.

In some embodiments of any of the aspects described above, the thermal insulating component comprises a steel surface. In some embodiments, the heat shield surface comprises a steel surface.

In some embodiments of any of the aspects described above, the tubular member or inner tubular member comprises a steel blade. Any of the tubular members may be made from steel. Steel is an advantageous material as it is very effective at cutting tissue. Therefore, if the tubular members, which may act as blades, comprise steel, they are very effective at cutting tissue. Further, stainless steel is a sterilisable material which is advantageous for surgical instruments.

From a fourth aspect, the present disclosure relates to an electrosurgical instrument comprising: an end effector according to any embodiments of the third aspect; and an operative shaft having RF electrical connections operably connected to the lateral facing radio frequency active electrode.

In some embodiments of the fourth aspect, the operative shaft further comprises drive componentry operably connected to a rotary shaver arrangement, the rotary shaver arrangement comprising the outer tubular member and the inner tubular member, to drive the rotary shaver arrangement to operate in use.

From a fifth aspect, the present disclosure relates to an electrosurgical system, comprising: an RF electrosurgical generator; a suction pump; and an electrosurgical instrument according to any embodiments of the fourth aspect, the arrangement being such that in use the RF electrosurgical generator supplies an RF coagulation or ablation signal via the RF electrical connections to the lateral facing radio frequency active electrode, and the suction pump supplies suction via the central suction lumen connecting the suction aperture located within the electrode to the suction pump.

From a sixth aspect, there is provided a method for processing an instrument for surgery, the method comprising: obtaining the rotary shaver component of the first aspect, the rotary shaver arrangement of the second aspect, or the end effector of the third aspect; sterilizing the rotary shaver component of the first aspect, the rotary shaver arrangement of the second aspect or the end effector of the third aspect; and storing the rotary shaver component of the first aspect, the rotary shaver arrangement of the second aspect or the end effector of the third aspect in a sterile container.

### BRIEF DESCRIPTION OF THE DRAWINGS

Embodiments of the invention will now be further described by way of example only and with reference to the accompanying drawings, wherein:

FIG. 1 is a CAD image showing a typical RF suction probe shaft construction (VAPR Tripolar 90);

FIG. 2 is a schematic diagram of an electrosurgical system including an electrosurgical instrument;

FIG. 3 is a CAD image showing the shaft construction of an RF shaver design concept;

FIG. 4 is a cross-sectional view of an RF shaver end effector illustrating an embodiment of the present invention;

FIG. 5 is a cross-sectional view of an RF shaver end effector illustrating an embodiment of the present invention;

FIG. 6 is a cross-sectional view of an RF shaver end effector illustrating an embodiment of the present invention;

FIG. 7 is a perspective view of a rotary shaver component (an inner blade) illustrating an embodiment of the present invention;

FIG. 8 is a perspective view of a rotary shaver component (an inner blade) illustrating an embodiment of the present invention;

FIG. 9 is an image of a rotary shaver component (an inner blade) with an embodiment of the present invention; and

FIG. 10 is a simplistic diagram of an embodiment of the present invention.

### DESCRIPTION OF THE EMBODIMENTS

An RF Shaver with opposite sided functionality (RF on one side, shaver on the other) must balance the RF functionality with the shaver functionality at the distal tip of the device. In this configuration, hot saline is created at the active tip and pulled via a negative pressure source (i.e., suction) into the distal tip assembly. In this assembly, the inner tubular member (also referred to as “inner blade”) is positioned in a closed position during RF use such that the cutting windows of the inner and outer tubular members do not overlap. The inner blade typically has a thin steel construction, which is very effective at cutting tissue, but not an effective thermal insulator.

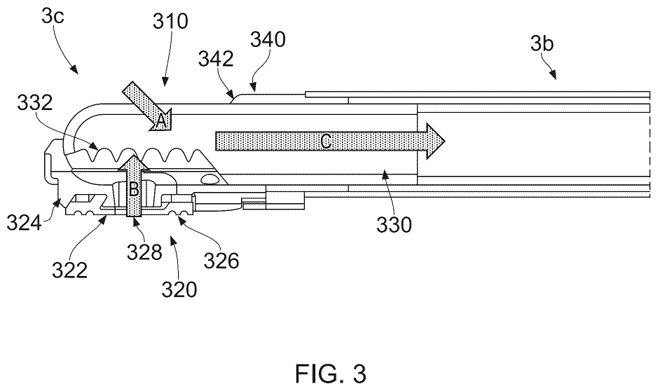

It is more difficult to implement thermal insulation into the RF shaver shaft design than in previous RF suction probes. This is because the outer shaft inner diameter is taken up almost entirely by the inner shaft, which is the main suction path for the heated saline. This results in a device construction shown in FIG. 3 that does not include the air gap and polymer insulation layers present on typical RF suction probes. This reduction in thermal insulation may leads to a greater risk of high outer shaft temperatures, which in turn may lead to risk of patient burn at the point of skin-contact.

Embodiments of the present invention provide a rotary shaver arrangement for a surgical instrument which has a thermal insulation component (i.e., a “heat shield”) which reduces the effect of the hot saline passing through the central suction lumen (which runs through the inner blade) on heating the inner blade, especially the part of the inner blade which comes into direct contact with non-target tissue on its outer surface.

The thermally insulating component is particularly useful for electrosurgical instruments which combine rotary shaver arrangements and RF electrode arrangements, where suction is used to remove RF heated saline from the surgical site. Without the presence of the thermally insulating component, there is a risk of burning the patient if the RF heated saline becomes too hot as the electrosurgical instrument may not be adequately insulated. The thermally insulating component prevents or reduces the effect of hot saline heating at least a portion of the inner tubular member, thereby preventing or reducing damage to non-target tissue during use of the radio frequency function.

In more detail, one example of an electrosurgical instrument within which the thermal insulating component may be advantageously integrated is a dual-sided RF shaver with suction capabilities. Such an instrument has a rotary arrangement of two concentric cylindrical shafts, an inner shaft (also referred to as an inner tubular member) and an outer shaft (also referred to as an outer tubular member). The inner shaft rotates relative to the outer shaft. Both the outer and inner shafts have a cutting window at their distal ends. Suction is applied along a suction path which extends from the proximal end of the instrument through the lumen of the inner shaft and through the cutting window. The suction draws tissue to be cut into the cutting window, where it is severed by the rotating inner shaft. The inner shaft may have serrated edges along its cutting window. The tissue is then suctioned into the lumen and taken away from the surgical site. In addition to the shaver capability, the instrument also has RF capabilities by virtue of a lateral facing RF electrode mounted on the opposite side of the operative shaft to the outer shaft's distal cutting window. The RF electrode may be used to cut, coagulate, desiccate or fulgurate tissue. Within the electrode is a suction aperture which is also connected to the lumen of the inner shaft. A suction path extends from the lumen through the suction aperture in the electrode. This suction path is an alternative to the suction path which extends through the cutting window of the shaver side. When the surgeon wishes to apply suction via the suction aperture on the RF side, the cutting window of the shaver side is closed by rotating the inner shaft such that the cutting windows of the inner and outer shaft are misaligned. The RF energy applied by the electrode may result in RF heated plasma which in turn heats saline and/or tissue. This heated material (saline and/or ablated tissue) is then suctioned away from the surgical site through the suction aperture within the electrode. The heated material therefore travels down the suction path through the lumen of the inner shaft. Without the presence of the thermal insulating component of embodiments of the present invention, this hot material may heat the outer shaft of the instrument to high temperatures. Such high temperatures could potentially burn the patient.

The Electrosurgical System

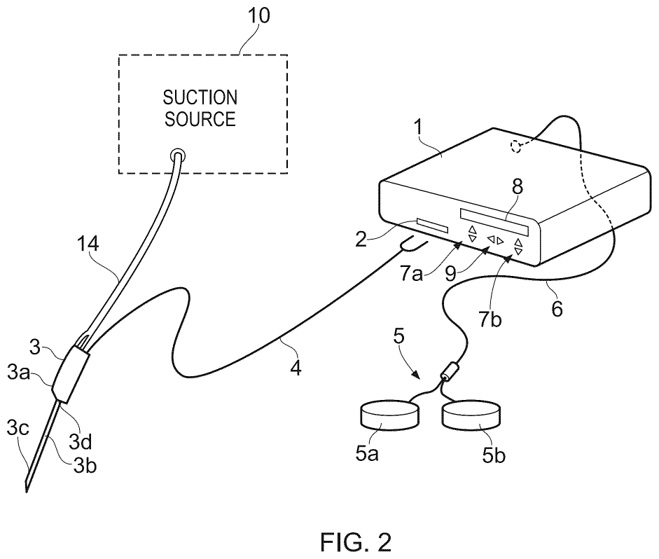

Referring to the drawings, FIG. 2 shows electrosurgical system including an electrosurgical generator 1 having an output socket 2 providing an RF output, via a connection cord 4, for an electrosurgical instrument 3. The instrument 3 has suction tubes 14 which are connected to a suction pump 10. Activation of the generator 1 may be performed from the instrument 3 via a handswitch (not shown) on the instrument 3, or by means of a footswitch unit 5 connected separately to the rear of the generator 1 by a footswitch connection cord 6. In the illustrated embodiment, the footswitch unit 5 has two footswitches 5a and 5b for selecting a coagulation mode or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes.

The Electrosurgical Instrument

The instrument 3 includes a proximal handle portion 3a, a hollow shaft 3b extending in a distal direction away from the proximal handle portion, and a distal end effector assembly 3c at the distal end of the shaft. A power connection cord 4 connects the instrument to the RF generator 1. The instrument may further be provided with activation buttons (not shown), to allow the surgeon operator to activate either the mechanical cutting function of the end effector, or the electrosurgical functions of the end effector, which typically comprise coagulation or ablation.

FIG. 3 shows an example of the distal end effector assembly 3c in more detail. The distal end effector 3c has two sides to it, the shaver side 310 and the RF side 320.

The inner shaft 330 is co-axially disposed within an outer shaft 340. The outer shaft 340 has a larger diameter than the inner shaft 330. The inner shaft 330 is a tubular member having a proximal end and a distal end, with cutting window 332 disposed at a side of its distal end. The outer shaft 340 is also a tubular member having a proximal end and a distal end, with cutting window 342 disposed at a side of its distal end. The inner shaft 330 is rotatably disposed inside of the outer shaft 340 such that the surgical instrument 3 cuts tissue by rotating the inner shaft 330 within the outer shaft 340 while a vacuum is applied through the lumen of the inner shaft 330 to draw the tissue into the cutting windows 332 and 342 and sever the tissue by rotation of the inner shaft.

The RF side 320 of the electrosurgical instrument 3 comprises an electrode assembly comprising an active electrode for tissue treatment (“active tip”) 322 received in a ceramic insulator 324. The active tip 322 is provided with projections 326 to concentrate the electric field at those locations. The projections 326 also serve to create a small separation between the planar surface of the active electrode 322 and the tissue to be treated. This allows conductive fluid to circulate over the planar surface and avoids overheating of the electrode or the tissue. The active tip 322 of the instrument is provided with a suction aperture 328, which is the opening to a lumen within an inner shaft 330.

In more detail, when the RF side 320 is to be used as a suction tool by applying a vacuum through the lumen within the inner shaft 330, the inner shaft 330 (which acts as a cutting blade) is stopped from rotating and the cutting windows 332 and 342 are misaligned with each other, i.e. closing the cutting windows, (as is the case in FIG. 3) so that the vacuum is applied through the suction path connecting the suction aperture 328 to the suction pump 10 via the lumen (i.e. the suction path defined by arrows B and C) to transport fluids to and from the active tip 322.

In contrast, when the shaver side 310 is in use for a cutting operation, suction flows via the suction path defined by arrows A and C, i.e. through the cutting windows to the lumen.

The inner and outer shafts 330 and 340 are made from a sterilisable material. For example, the sterilisable material may be a metal such as stainless steel.

Thermal Insulation Component

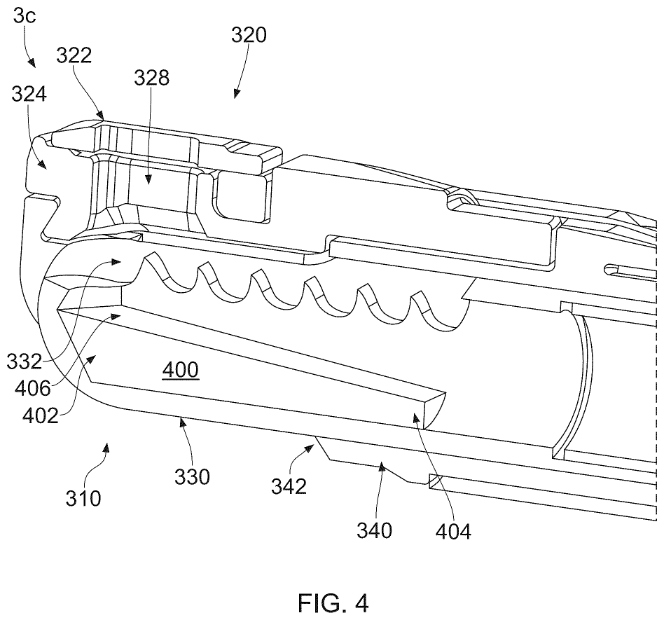

Embodiments of the present invention provide a rotary shaver component, a rotary shaver arrangement, and an end effector comprising a thermal insulation component. Embodiments of the present invention will be described in more detail with reference to FIGS. 4 to 9.

FIGS. 4-9 show a distal end effector assembly 3c which is similar to that shown in FIG. 3, like reference numerals are used accordingly. The end effector assemblies of FIGS. 4-9 differ from that of FIG. 3 in that FIGS. 4-9 additionally show a thermal insulation component 400 which is the subject of the present disclosure. The thermal insulation component or “heat shield” 400 is located on the inner surface of the inner blade 330.

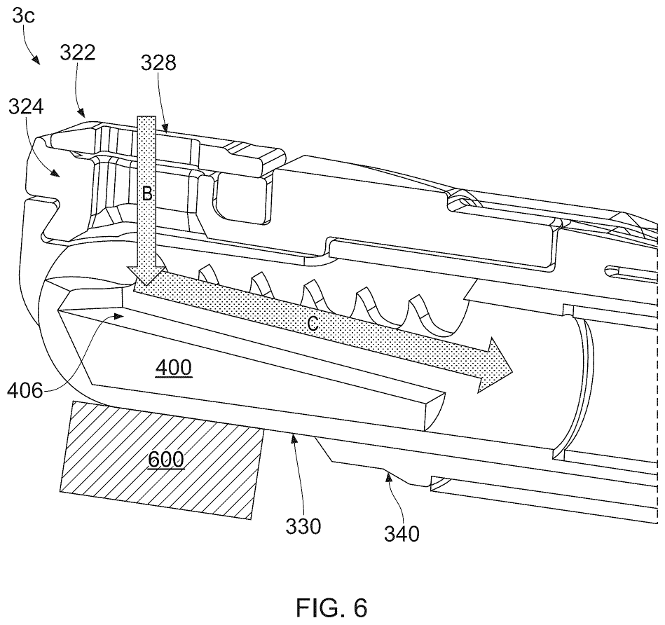

FIG. 6 shows in more detail the path hot saline takes from the suction aperture 328 via path B, then through the central suction lumen via path C during usage of the RF function. With reference to FIG. 6, when the RF side 320 of the instrument is in use, i.e., the RF function is active, the inner tubular member 330 rotates such that the cutting window 332 of the inner tubular member 330 is facing up towards the RF side 320, thereby misaligning the cutting window 332 of the inner tubular member with the cutting window of the outer tubular member 342. This seals the central suction lumen C such that the only open aperture in communication with the central suction lumen C is the RF aperture 328. This results in adequate suction from the surgical site which the RF side 320 is acting on. When the RF side 320 is in use and the inner blade 330 is in this first position, the portion of the inner blade 330 which is opposite the cutting window 332 is often in direct contact with non-target tissue 600. It is the outer surface of the inner blade 330 which is in contact with the non-target tissue 600. When the hot saline is suctioned from the RF surgical site, through the RF aperture 328 and along the central suction lumen C, the hot saline would ordinarily transfer heat energy to the inner blade 330, and thus the outer surface of the inner blade 330 may get dangerously hot and potentially burn the patient. The presence of the thermally insulating heat shield 400 at this position on the inner surface of the inner tubular member 330 opposite the cutting window 332 reduces the transfer of heat energy to the inner blade 330 at this position, thereby reducing the likelihood of burns to the non-target tissue 600. Were the heat shield 400 not present, the hot saline would heat the part of the inner blade which may be in direct contact with the non-target tissue 600. This could result in burns to the patient. The presence of the heat shield 400 results in reduced or no heating of the part of the inner blade which may be in direct contact with the non-target tissue 600.

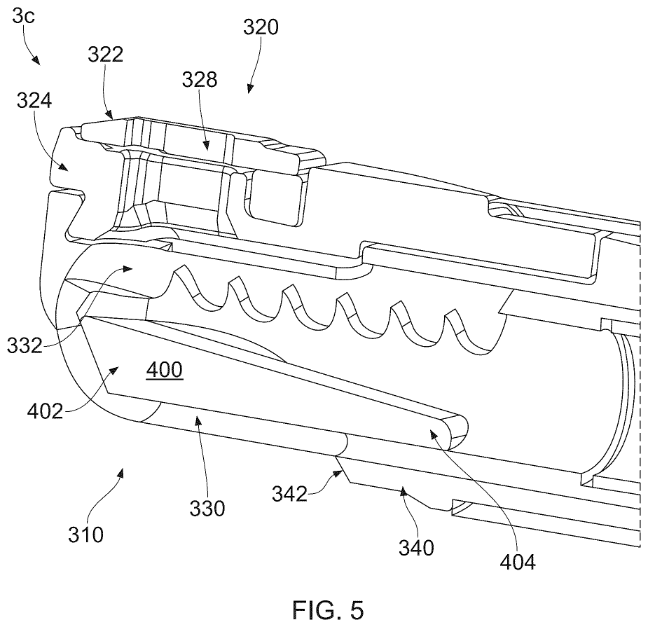

The geometry of the heat shield 400 shown in the Figures is exemplary only, for example, a thermally insulating coating (e.g., a thick conformal coating) of insulator material could provide the same effect. In the example shown in FIGS. 4-9, the heat shield 400 is arranged such that the distal end 402 of the heat shield/thermal insulation component 400 is thicker than the proximal end 404 of the heat shield/thermal insulation component 400. This is advantageous as it provides sufficient thermal insulation where needed while leaving adequate space for cut tissue to be suctioned along the central suction lumen and thus removed from the surgical site. In the examples shown in FIGS. 4-9, the heat shield 400 is shaped such that it provides a sloped surface from the distal end of the inner tubular member 330 such that the height of the heat shield 400 is greater at the distal end 402 of the heat shield 400 than at the proximal end 404 of the heat shield 400. In FIG. 5, the heat shield 400 is shaped such that the width of the heat shield 400 is greater at the distal end of the heat shield 400 than at the proximal end of the heat shield 400. Both of these arrangements (changing width and/or changing height) provide a heat shield 400 which provides sufficient thermal insulation where needed while leaving adequate space for cut tissue to be suctioned along the central suction lumen and thus removed from the surgical site.

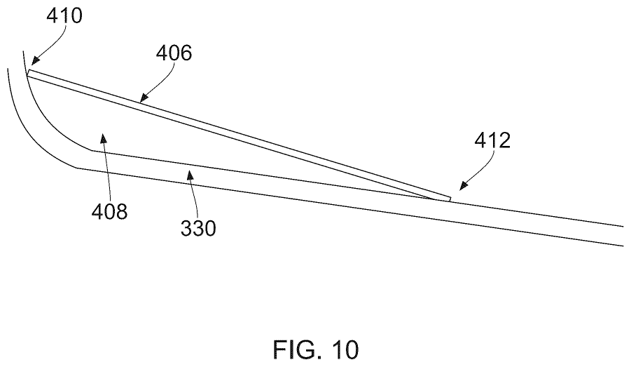

The heat shield 400 can comprise any sufficient material with good thermal properties and an ability to be fixed within the inner blade. For example, a ceramic such as Alumina or Zirconia, or any blend could be used for the heat shield 400. Alternatively or additionally, various heat resistant polymers could be used for the heat shield 400. The thickness of the heat shield 400 would be chosen as a function of its thermal properties (conductivity and thermal heat capacity), for example, if the material is a good insulator, the heat shield can be thinner while providing adequate thermal insulation. The heat shield 400 may be a solid or a hollow component. The example shown in FIGS. 4-9 is a solid component, but the features and description of the solid component apply to a hollow component (example shown in FIG. 10) accordingly.

The heat shield 400 could be fitted via interference fit, adhesive, snap fits or any other suitable method for holding the component in the required location.

In embodiments where the heat shield is a hollow component, the heat shield may comprise a heat shield surface 406 and an air gap 408 such that the heat shield surface 406 encloses a pocket of air (the air gap 408) behind the heat shield surface 406. This is illustrated in a simplistic diagram shown in FIG. 10. This is advantageous because air 408 is a better thermal insulator than most solid materials, and air is also lighter than a solid material so would not upset the rotational balance of the blade as much when spinning. The heat shield surface 406 may have the same geometry as described above in relation to the figures (i.e., the heat shield surface would be the same as the upper surface 406 (see FIG. 4) of heat shield 400, but with an air gap 408 behind the shield instead of bulk material. The heat shield surface 406 may be made from steel. The heat shield surface 406 may be welded to the inner surface of the inner tubular member 330, e.g., a first end 410 of the heat shield surface 406 may be welded to the inner surface and a second end 412 of the heat shield surface 406 may be welded to the inner surface, thereby leaving an air gap 408 between the inner surface and the heat shield surface to act as an insulator.

The key is that the heat shield 400 is located on at least a portion of the inner surface of the inner tubular member 330 to prevent or reduce heat from the hot saline being transferred to the part of the inner tubular member 330 which may be in direct contact with non-target tissue 600.

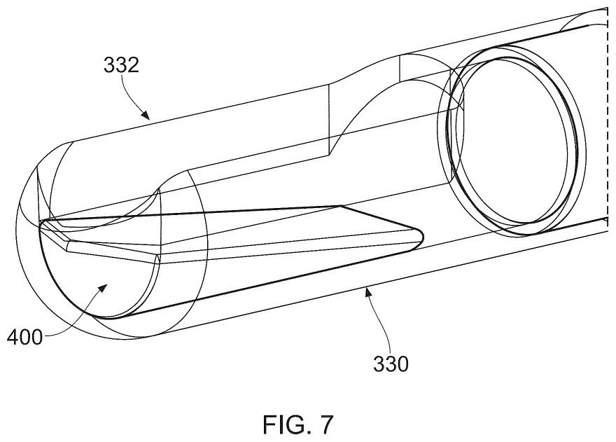

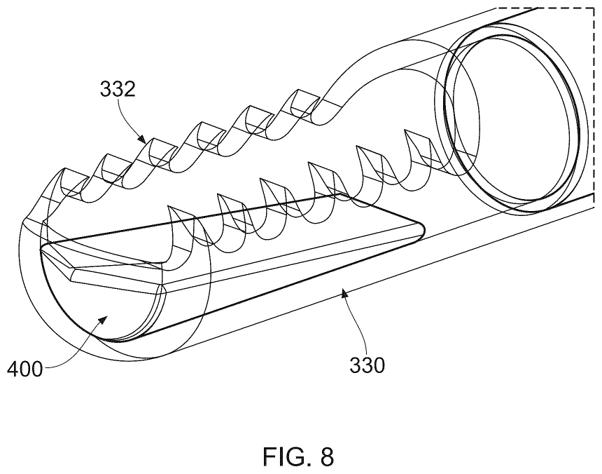

The heat shield 400 can be easily adapted to fit various inner blade tooth styles as shown in FIGS. 7 and 8. FIGS. 7 and 8 only show the inner blade 330 (i.e., they do not show the outer blade or the RF side of the instrument). In FIG. 7, the cutting window 332 of the inner blade 330 has a straight-edge blade and the distal end of the cutting window 332 slopes downwards to the distal end. In FIG. 8, the cutting window 332 of the inner blade 330 has a serrated blade and the distal end of the cutting window 332 slopes upwards to the distal end. The geometry of the heat shield 400 can be adapted to fit both of these arrangements, and similarly can be adapted for other alternative rotary shaver arrangements as necessary.

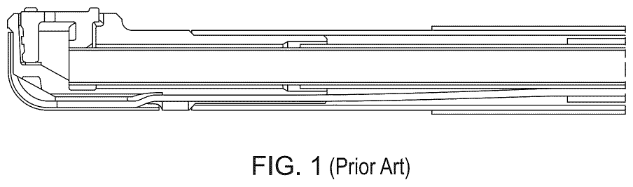

Further, the heat shield of the present invention may be used in RF probes which do not have rotary shaver functions. For example, the thermal insulation properties of the typical RF probe shown in FIG. 1 could be improved by providing the heat shield of the present invention on the inner surface of the tubular member opposite the lateral RF electrode. This could be in addition to the insulation provided beyond the outer surface of the tubular member.

The presence of the thermal insulating component/heat shield 400 results in a substantial reduction in the inner blade temperature in an RF shaver configuration.

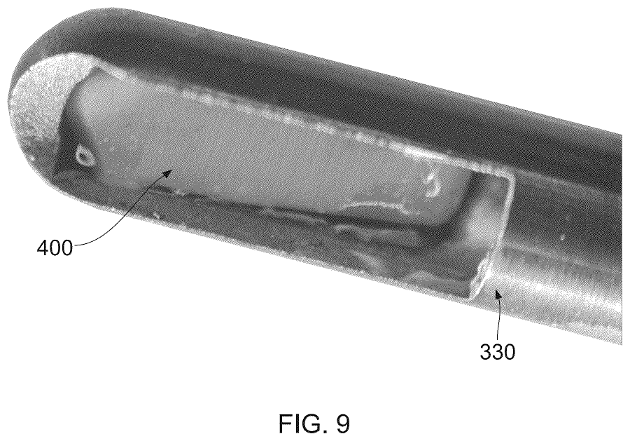

FIG. 9 shows an image of one example of the present invention, a Zirconia insulator bonded with adhesive into a steel inner blade.

Reprocessing

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The device may also be sterilized using any other technique known in the art, including but limited to beta or gamma radiation, ethylene oxide, or steam.

Various modifications whether by way of addition, deletion, or substitution of features may be made to above-described embodiment to provide further embodiments, any and all of which are intended to be encompassed by the appended claims.

1. An end effector for an electrosurgical instrument, the end effector comprising:
a tubular member providing a central suction lumen;
a lateral facing radio frequency active electrode mounted at the distal end of the tubular member, the electrode comprising a suction aperture, the suction aperture being in fluid communication with the central suction lumen;
a thermal insulation component provided at the distal end of the tubular member, the thermal insulation component being provided on at least a portion of an inner surface of the tubular member, wherein, when the radio frequency active electrode is in use, the portion of the inner surface is located opposite the lateral facing radio frequency active electrode.

2. The end effector of claim 1, the end effector further comprising an outer tubular member having a central passageway; wherein the tubular member is an inner tubular member rotatably mounted in the central passageway of the outer tubular member, the inner tubular member comprises a first cutting window at the distal end thereof, the outer tubular member comprises a second cutting window at the distal end thereof, such that the first and second cutting windows align when the inner tubular member is rotated to a first position, and wherein the portion of the inner surface is located opposite the first cutting window.

3. The end effector of claim 2, wherein the arrangement is such that, when in use, rotation of the inner tubular member within the outer tubular member causes a tissue cutting action of the first cutting window interacting with the second cutting window.

4. The end effector of claim 1, wherein the thermal insulation component is such that the distal end of the thermal insulation component is thicker than the proximal end of the thermal insulation component.

5. The end effector of claim 4, wherein the thermal insulation component is shaped such that it provides a sloped surface from the distal end of the inner tubular member such that the height of the thermal insulation component is greater at the distal end of the thermal insulation component than at the proximal end of the thermal insulation component.

6. The end effector of claim 1, wherein the thermal insulation component comprises a thermally insulating coating on at least the portion of the inner surface of the inner tubular member.

7. The end effector of claim 1, wherein the thermal insulation component comprises a ceramic material.

8. The end effector of claim 7, wherein the ceramic material comprises Alumina and/or Zirconia.

9. The end effector of claim 1, wherein the thermal insulation component comprises a heat resistant polymer.

10. The end effector of claim 1, wherein the thermal insulation component is fitted to at least the portion of the inner surface by one or more of the following: interference fit, adhesive and snap fit.

11. The end effector of claim 1, wherein the thermal insulating component is a solid component.

12. The end effector of claim 1, wherein the thermal insulating component is a hollow component.

13. The end effector of claim 1, wherein the thermal insulating component comprises a heat shield surface and an air gap, wherein the air gap is positioned between the heat shield surface and the inner surface of the tubular member located opposite the first cutting window.

14. The end effector of claim 12, wherein the thermal insulation component is fitted to at least the portion of the inner surface by welding.

15. The end effector of claim 12, wherein the thermal insulating component comprises a steel surface.

16. The end effector of claim 1, wherein the tubular member or inner tubular member comprises a steel blade.

17. An electrosurgical instrument comprising:
an end effector according to claim 1; and
an operative shaft having RF electrical connections operably connected to the lateral facing radio frequency active electrode.

18. The electrosurgical instrument of claim 17, the end effector further comprising an outer tubular member having a central passageway; wherein the tubular member is an inner tubular member rotatably mounted in the central passageway of the outer tubular member, the inner tubular member comprises a first cutting window at the distal end thereof, the outer tubular member comprises a second cutting window at the distal end thereof, such that the first and second cutting windows align when the inner tubular member is rotated to a first position, and wherein the portion of the inner surface is located opposite the first cutting window, the operative shaft further comprising drive componentry operably connected to a rotary shaver arrangement, the rotary shaver arrangement comprising the outer tubular member and the inner tubular member, to drive the rotary shaver arrangement to operate in use.

19. A rotary shaver component for a surgical instrument, the rotary shaver component comprising:
a tubular member providing a central suction lumen, the tubular member comprising a first cutting window at the distal end thereof; and
a thermal insulation component provided at the distal end of the tubular member, the thermal insulation component being provided on at least a portion of an inner surface of the tubular member located opposite the first cutting window.

20. A rotary shaver arrangement for a surgical instrument, the rotary shaver arrangement comprising:
an outer tubular member having a central passageway, the tubular member comprising a second cutting window at the distal end thereof; and
a rotary shaver component for a surgical instrument, the rotary shaver component comprising: a tubular member providing a central suction lumen, the tubular member comprising a first cutting window at the distal end thereof; and a thermal insulation component provided at the distal end of the tubular member, the thermal insulation component being provided on at least a portion of an inner surface of the tubular member located opposite the first cutting window;
wherein the tubular member is an inner tubular member rotatably mounted in the central passageway of the outer tubular member and the first and second cutting windows align when the inner tubular member is rotated to a first position.
